(19) United States
(12) Patent Application Publication
(10) Pub. No.: US 2026/0183417 A1
(43) Pub. Date: Jul. 2, 2026

(54) CARDIAC-TARGETING EXTRACELLULAR VESICLE (EV), EV FOR TREATING CARDIOVASCULAR DISEASE, AND METHOD OF PREPARATION AND USE THEREOF

(71) Applicant: Shanghai University, Shanghai (CN)

(72) Inventors: Junjie XIAO, Shanghai (CN); Jin LI, Shanghai (CN); Xiaohang YIN, Shanghai (CN); Hongjian CHEN, Shanghai (CN)

(73) Assignee: Shanghai University, Shanghai (CN)

(21) Appl. No.: 19/392,212

(22) Filed: Nov. 18, 2025

(30) Foreign Application Priority Data
Dec. 30, 2024 (CN) 202411975722.9

(51) Int. Cl. A61K 47/69; A61K 9/50; A61K 9/51; A61P 9/00; C07K 7/08; C07K 14/705; C12N 15/113; C12N 15/62; C12N 15/85

(52) U.S. Cl. CPC A61K 47/6901; A61K 9/5068; A61K 9/5192; A61P 9/00; C07K 7/08; C07K 14/70596; C12N 15/113; C12N 15/62; C12N 15/85; C07K 2319/00; C12N 2310/141

(57) ABSTRACT

A cardiac-targeting extracellular vesicle (EV), an EV for treating cardiovascular disease, and method of preparation and use thereof are provided. The EV contains a fusion sequence of Lamp-2b and peptide targeting cardiacmyocytes, CMP. It has been discovered that the cardiac-targeting EV exhibits the ability to specifically target the heart, thereby serving as a novel delivery tool system for cardiac-targeting therapy. The cardiac-targeting EV is loaded with miR-30d to obtain an EV for treating the cardiovascular disease, which demonstrates significant therapeutic effects on conditions such as cardiac ischemia-reperfusion injury and myocardial hypertrophy. This provides a new therapeutic approach for the clinical treatment of cardiovascular diseases. Additionally, targeted delivery of therapeutic agents using the cardiac-targeting EV is achieved, enabling effective prevention and treatment of cardiac diseases.

### CROSS-REFERENCE TO THE RELATED APPLICATION

This application is based upon and claims priority to Chinese Patent Application No. 202411975722.9, filed on Dec. 30, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0299_SequenceListing.xml, created on Oct. 17, 2025, and is 25,890 bytes in size.

### TECHNICAL FIELD

The present disclosure belongs to the technical field of biomedicine, and in particular relates to a cardiac-targeting extracellular vesicle (EV), an EV for treating cardiovascular disease, and a method of preparation and use thereof.

### BACKGROUND

Cardiovascular diseases (CVDs) are the leading cause of global mortality. With the aging population and the widespread prevalence of risk factors associated with cardiovascular diseases, the incidence of these diseases is expected to rise. Among them, conditions such as cardiac ischemia-reperfusion injury and myocardial hypertrophy have high morbidity rates and lack effective treatment options.

Extracellular vesicles (EVs) are endogenous membrane-bound nanoparticles secreted by cells, containing proteins, nucleic acids, lipids, and other substances. They play a role as delivery vehicles in intercellular communication and various biological processes. Because EVs are endogenous biomolecules, they exhibit low immunogenicity and good safety profiles. However, natural EVs lack targeting capability, and imparting targeting ability to EVs is crucial for achieving targeted delivery. Currently, targeting modifications on the surface of EVs can be categorized into biological modifications and chemical modifications. Biological modifications involve fusing specific peptide sequences or ligand sequences into the surface of EVs through genetic engineering, thereby endowing the EVs with targeting peptides, which compensates for the natural lack of targeting in EVs. Chemical modifications involve using natural or synthetic ligands to modify the surface of EVs through coupling reactions or lipid assembling methods. However, this approach can be affected by the complex structure of the membrane surface, leading to lower modification efficiency, and the assembling methods may disrupt the membrane structure of the EVs.

MicroRNAs (miRNAs) are a class of widely expressed endogenous non-coding RNA molecules, approximately 22 nucleotides in length. MiRNAs can participate in many biological processes by imperfectly pairing with mRNA sequences, thereby inhibiting mRNA translation or leading to transcript degradation. In the field of cardiovascular research, miRNAs have been shown to be associated with cardiovascular diseases such as myocardial infarction, myocardial hypertrophy, and arrhythmias. However, there are currently no reports on the preparation of cardiac-targeting EVs using miRNAs.

### SUMMARY

In light of this, an objective of the present disclosure is to provide a cardiac-targeting extracellular vesicle (EV), an EV for treating cardiovascular disease, and a method of preparation and use thereof. The cardiac-targeting EV in the present disclosure serves as a targeted delivery tool for the treatment of cardiovascular disease, and by loading miR-30d, it achieves targeted delivery of miR-30d to the heart, thereby providing a therapeutic effect for cardiovascular diseases.

To achieve the above objective, the present disclosure provides the following technical solutions.

The present disclosure provides a cardiac-targeting EV including a fusion sequence of lysosome-associated membrane protein 2, isoform b (Lamp-2b) and cardiacmyocyte-specific peptide (CMP); the fusion sequence of the Lamp-2b and the peptide targeting cardiacmyocytes, CMP, is set forth in SEQ ID NO: 11.

The present disclosure further provides a method for preparing a cardiac-targeting EV, including the following steps:
inserting a sequence of the peptide targeting cardiacmyocytes, CMP, into a plasmid-cDNA (pcDNA) GNSTM-3-rabies virus glycoprotein (RVG)-10-Lamp2b-human influenza hemagglutinin (HA) plasmid to obtain a recombinant plasmid, transfecting the recombinant plasmid into a host cell to obtain a recombinant cell, and culturing the recombinant cell to secrete the cardiac-targeting EV, and isolating the cardiac-targeting EV, where
the host cell is a human embryonic kidney 293 cell expressing the SV40 large T antigen (HEK293T) cell.

In some embodiments, the nucleotide sequence encoding the peptide targeting cardiacmyocytes, CMP, is set forth in SEQ ID NO: 12 and the amino acid sequence of the peptide targeting cardiacmyocytes, CMP, is set forth in SEQ ID NO: 13.

In some embodiments, a ratio of the recombinant plasmid to the host cell is 8-12 μg: 2.8-3.2 million cells.

The present disclosure further provides use of the cardiac-targeting EV or the method of preparation in preparation of a cardiac-targeting product.

The present disclosure further provides an EV for treating cardiovascular disease, which is obtained by loading the cardiac-targeting EV with miR-30d.

The present disclosure further provides a method for preparing an EV for treating the cardiovascular disease, including the following steps:

inserting a sequence of the peptide targeting cardiacmyocytes, CMP, into a pcDNA GNSTM-3-RVG-10-Lamp2b-HA plasmid to obtain a recombinant plasmid, transfecting the recombinant plasmid into a host cell to obtain a recombinant cell, and culturing the recombinant cell to secrete the EV, and isolating the EV for treating the cardiovascular disease, where
the host cell is a stable miR-30d-transfected HEK293T cell;
the stable miR-30d-transfected HEK293T cell is prepared by co-transfecting a plasmid that overexpresses miR-30d with a packaging plasmid into the HEK293T cell.

The present disclosure further provides use of the above EV for treating cardiovascular disease or the above method of preparation in preparation of a drug for treating the cardiovascular disease.

The present disclosure further provides use of the above EV for treating the cardiovascular disease or the above method of preparation in preparation of a targeted drug for treating the cardiovascular disease.

The present disclosure further provides a drug for treating cardiovascular disease, which includes the above EV or an EV obtained from the above method of preparation, along with pharmaceutically acceptable excipient.

Compared to the prior art, embodiments of the present disclosure has the following beneficial effects.

The present disclosure provides a cardiac-targeting EV, an EV for treating cardiovascular disease, and a method of preparation and use thereof. In the present disclosure, the cardiac-targeting peptide is modified onto the surface of the EV through the Lamp-2b protein, resulting in a cardiac-targeting EV. Research has shown that the cardiac-targeting EV has a targeting effect on the heart. This cardiac-targeting EV may serve as a novel delivery tool for cardiac-targeting therapy. By loading miR-30d, the cardiac-targeting EV results in an EV for treating the cardiovascular disease, which shows significant therapeutic effects on cardiovascular diseases such as cardiac ischemia-reperfusion injury and myocardial hypertrophy, providing a new treatment approach for clinical treatment of cardiovascular diseases. Additionally, in the present disclosure cardiac-targeting EVs are utilized for targeted delivery of therapeutic drugs, achieving the goal of effectively preventing and treating the cardiac diseases.

### BRIEF DESCRIPTION OF THE DRAWINGS

FIG. 1 is a schematic diagram illustrating the principle of constructing fusion plasmids through homologous recombination.

FIG. 2 is a schematic diagram of the pcDNA GNSTM-3-RVG-10-Lamp2b-HA plasmid structure.

FIG. 3 is an image illustrating the agarose gel electrophoresis of the linearized product amplified by polymerase chain reaction (PCR) from the plasmid vector.

FIG. 4 is an image illustrating the agarose gel electrophoresis of the homologous recombination ligation product.

FIG. 5 shows sequencing results of the genes of interest (GOIs) inserted into pcDNA GNSTM-3-EGFP-10-Lamp2b-HA (SEQ ID NOS: 19-20) and pcDNA GNSTM-3-CMP-10-Lamp2b-HA (SEQ ID NO: 12), respectively.

FIG. 6 is a flowchart of the preparation process for cardiac-targeting extracellular vesicles (EVs) using differential centrifugation.

FIG. 7 shows results of particle size detection for EVs-EGFP and EVs-CMP extracellular vesicles using a nanoparticle tracking analyzer.

FIG. 8 shows results of immunoblotting for detecting surface marker proteins of EVs-EGFP and EVs-CMP extracellular vesicles.

FIG. 9 shows results of uptake of EVs-EGFP and EVs-CMP extracellular vesicles by H9C2, HepG2, BEAS-2B, and HEK293T cells.

FIG. 10 shows results of flow cytometry detecting the uptake of EVs-EGFP and EVs-CMP extracellular vesicles by H9C2 cells, with ***p<0.001.

FIG. 11A-FIG. 11B show enrichment of phosphate-buffered saline (PBS), EVs-EGFP, and EVs-CMP extracellular vesicles in mouse hearts, where the left panel shows fluorescence images of the enrichment, while the right panel shows the radiant efficiency of PBS, EVs-EGFP, and EVs-CMP in mouse hearts, with ***p<0.001.

FIG. 12 shows results of real-time quantitative reverse transcription polymerase chain reaction (qRT-PCR) detecting the expression level of miR-30d in stable-transfected cell lines, with ***p<0.001.

FIG. 13 shows results of qRT-PCR detecting the expression levels of miR-30d in EVs-CMP-Scramble, EVs-CMP-miR-30d, and EVs-EGFP-miR-30d extracellular vesicles, with **p<0.01.

FIG. 14 shows transmission electron microscopy results showing the size and morphology of EVs-CMP-miR-30d and EVs-EGFP-miR-30d extracellular vesicles.

FIG. 15 shows results of real-time quantitative PCR detecting miR-30d levels in heart tissues after different treatments, with **p<0.01.

FIG. 16 shows fluorescence images from immunofluorescence staining detecting apoptosis levels in cardiomyocytes.

FIG. 17 shows statistical results of immunofluorescence staining detecting apoptosis levels in cardiomyocytes, with ***p<0.001.

FIG. 18A-FIG. 18B show results of immunoblotting detecting levels of apoptosis-related proteins Bax/Bcl2 and Caspase3 after treatment with EVs-CMP-miR-30d and EVs-EGFP-miR-30d extracellular vesicles, with ***p<0.001.

FIG. 19 shows detection results of miR-30d levels in heart tissues after treatment with different extracellular vesicles, with **p<0.01, ***p<0.001.

FIG. 20A-FIG. 20C show results of echocardiography detecting cardiac function in mice after treatment with different extracellular vesicles, where FIG. 20A shows static images from echocardiography; FIG. 20B shows ejection fraction (EF) measured by echocardiography; and FIG. 20C shows fractional shortening (FS) measured by echocardiography, with ***p<0.001.

FIG. 21A-FIG. 21B show results of Masson staining detecting fibrosis levels in mouse hearts after treatment with different extracellular vesicles, where FIG. 21A shows Masson staining images of mouse heart tissues; and FIG. 21B shows statistical results for the fibrosis area after treatment with different extracellular vesicles, with ***p<0.001.

FIG. 22 is an experimental flowchart illustrating the effects of extracellular vesicles on cardiac hypertrophy.

FIG. 23 shows miR-30d levels in heart tissues as detected after treatment with cellular vesicles in mice with cardiac hypertrophy, with **p<0.01.

FIG. 24A-FIG. 24C show results of echocardiography detecting the effects of extracellular vesicle injection on heart function in hypertrophic mice, where FIG. 24A shows static images from echocardiography; FIG. 24B shows ejection fraction (EF) measured by echocardiography; and FIG. 24C shows fractional shortening (FS) measured by echocardiography, with ***p<0.001.

FIG. 25A shows results of cross-sectional area sizes of cardiomyocytes after treatment with different extracellular vesicles; and FIG. 25B shows statistical results of cross-sectional area sizes of cardiomyocytes after treatment, with *p<0.05, ***p<0.001.

FIG. 26A-FIG. 26B show results of Masson staining detecting fibrosis levels in mouse hearts after treatment with different extracellular vesicles, with *p<0.05, ***p<0.001.

FIG. 27A-FIG. 27B show results of hematoxylin-eosin (HE) staining detecting the effects on mouse cardiomyocytes after treatment with different extracellular vesicles, with ***p<0.001.

FIG. 28A-FIG. 28B show results of wheat germ agglutinin (WGA) staining detecting the effects on mouse heart size after treatment with different extracellular vesicles, with ***p<0.001.

### DETAILED DESCRIPTION OF THE EMBODIMENTS

The present disclosure provides a cardiac-targeting extracellular vesicle (EV) including a fusion sequence of Lamp-2b and peptide targeting cardiacmyocytes, CMP; and the fusion sequence of Lamp-2b and peptide targeting cardiacmyocytes, CMP, is set forth in SEQ ID NO: 11.

In the present disclosure, the N-terminal of the Lamp-2b on the surface of the extracellular vesicle is modified to include the sequence of the peptide targeting cardiacmyocytes, CMP, thereby imparting cardiac-targeting properties to the vesicle.

The present disclosure further provides a method for preparing the cardiac-targeting EV, which includes the following steps:

inserting a sequence of the peptide targeting cardiacmyocytes, CMP, into a pcDNA GNSTM-3-RVG-10-Lamp2b-HA plasmid to obtain a recombinant plasmid, transfecting the recombinant plasmid into a host cell to obtain a recombinant cell, and culturing the recombinant cell to secrete the the cardiac-targeting EV, and isolating the cardiac-targeting EV, where the host cell is HEK293T cell.

The cardiac-targeting extracellular vesicles of the present disclosure are achieved through genetic modification.

In the present disclosure, the sequence of the peptide targeting cardiacmyocytes, CMP, is inserted into the pcDNA GNSTM-3-RVG-10-Lamp2b-HA plasmid to obtain the recombinant plasmid. The fusion sequence of Lamp-2b and peptide targeting cardiacmyocytes, CMP, is set forth in SEQ ID NO: 11. The peptide targeting cardiacmyocytes, CMP, has the nucleotide sequence of SEQ ID NO: 12, and the amino acid sequence of SEQ ID NO: 13. The insertion of the sequence of peptide targeting cardiacmyocytes, CMP, involves replacing the rabies virus glycoprotein (RVG) fragment. In the present disclosure, the recombinant plasmid is also referred to as a fusion plasmid, abbreviated as pcDNA GNSTM-3-CMP-10-Lamp2b-HA plasmid. The pcDNA GNSTM-3-RVG-10-Lamp2b-HA plasmid used in the present disclosure is purchased from Addgene, catalog No. 71294.

In the present disclosure, after the recombinant plasmid is obtained, it is transfected into host cells to obtain a recombinant cell. The transfection is performed using polyethyleneimine (PEI) as a transfection agent, and the transfection time is preferably 10 to 14 hours, more preferably 12 hours. The host cell is human embryonic kidney 293 cell expressing the SV40 large T antigen (HEK293T) cell, which is a kind of human embryonic kidney cell. There are no specific limitations on the source of the HEK293T cell in the present disclosure; and commercially available products known in the field can be used. The ratio of the recombinant plasmid to the host cell is preferably 8 to 12 μg: 2.8 to 3.2 million cells, more preferably 9 to 11 μg: 2.9 to 3.1 million cells, and most preferably 10 μg: 3 million cells.

In the present disclosure, after the recombinant cell is obtained, the recombinant cell is cultured to secrete the cardiac-targeting EV, and the cardiac-targeting EV is isolated. The culture is performed for 48 hours using a high-glucose Dulbecco's modification of Eagle's medium (DMEM) containing 10% fetal bovine serum (FBS) but no EVs (purchased from Corning). The preferred method for isolation is differential centrifugation. Specifically, after 48 hours of culture, the cells are centrifuged at 300×g for 10 minutes to collect supernatant a. The collected supernatant a is then centrifuged at 4° C., 300×g for 10 minutes to remove viable cells, and supernatant b is collected. To remove dead cells, supernatant b is placed in a new centrifuge tube and centrifuged at 4° C., 2000×g for 10 minutes to obtain supernatant c. Supernatant c is then placed in a new centrifuge tube and centrifuged at 4° C., 10000×g for 30 minutes to remove cell debris, obtaining supernatant d. Finally, supernatant d is subjected to ultracentrifugation at 4° C., 100000×g for 70 minutes to obtain a pellet, which results in the isolation of cardiac-targeting EVs.

Through research, it has been found that the cardiac-targeting EV in the present disclosure possesses cardiac targeting functions. Therefore, the present disclosure further provides use of the cardiac-targeting EV or the method of preparation thereof in the manufacture of a product targeting the heart.

In the present disclosure, the product includes a reagent, a kit, or a drug.

The present disclosure further provides an EV for treating cardiovascular disease, which is obtained by loading the cardiac-targeting EV with miR-30d.

In the present disclosure, the EV for treating cardiovascular disease is also referred to as miR-30d-loaded cardiac-targeting EV.

The present disclosure further provides a method for preparing the EV for treating the cardiovascular disease, including the following steps:
inserting a sequence of the peptide targeting cardiacmyocytes, CMP, into a pcDNA GNSTM-3-RVG-10-Lamp2b-HA plasmid to obtain a recombinant plasmid, transfecting the recombinant plasmid into a host cell to obtain a recombinant cell, culturing the recombinant cell to secrete the EV, and isolating the EV for treating the cardiovascular disease, where
the host cell is a stable miR-30d-transfected HEK293T cell.

The stable miR-30d-transfected HEK293T cell is prepared by co-transfecting an HEK293T cell with a plasmid that overexpresses miR-30d and a lentiviral packaging plasmid.

In the present disclosure, a backbone plasmid for overexpressing miR-30d is preferably the pLKO.1-TRC vector, and the lentiviral packaging plasmid can be selected from pSPAX2 and pMD2.G. The mass ratio of the plasmid that overexpresses miR-30d, pSPAX2, and pMD2.G in the present disclosure is 4:3:1.

The present disclosure further provides use of the EV for treating the cardiovascular disease or the method of preparation thereof in the manufacture of a drug for treating the cardiovascular disease.

The present disclosure further provides use of the EV for treating the cardiovascular disease or the method of preparation thereof in the manufacture of a targeted drug for treating the cardiovascular disease.

In the present disclosure, the cardiovascular disease includes one or more of heart failure and cardiac hypertrophy, with heart failure being induced by acute ischemia-reperfusion injury.

The present disclosure further provides a drug for treating cardiovascular disease, the drug including the above EV or the EV obtained by the method of preparation thereof, and a pharmaceutically acceptable excipient.

In the present disclosure, an active ingredient of the drug includes the EV for treating the cardiovascular disease, and the excipient may include one or more of a diluent, a filler, a flavoring agent, a preservative, and a surfactant. The mass percentage of the EV for treating the cardiovascular disease in the drug is 30% to 70%.

In the present disclosure, unless otherwise specified, the raw materials or components used are all well-known products or commercially available products in the field.

The following detailed description of the technical solutions provided by the present disclosure is based on specific examples but should not be understood as a limitation of the protection scope of the present disclosure.

### Example 1

A method for preparing cardiac-targeting EVs is described, which included the following steps:

### 1. Construction of a Fusion Plasmid Containing the Peptide Targeting Cardiacmyocytes, CMP, Nucleic Acid Sequence and the Lamp-2b Nucleic Acid Sequence.

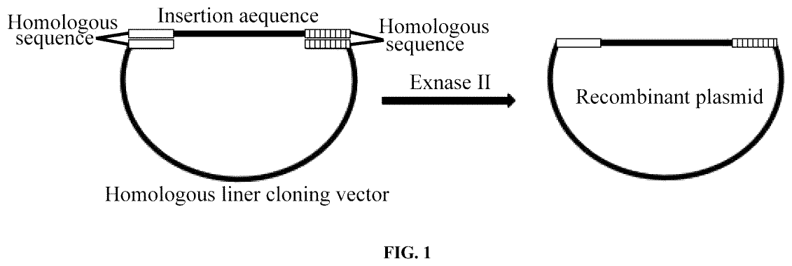

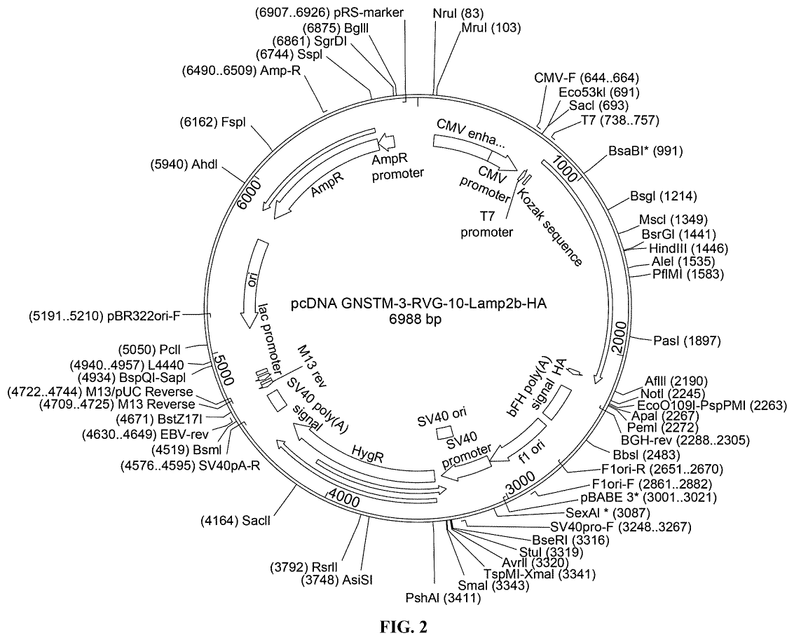

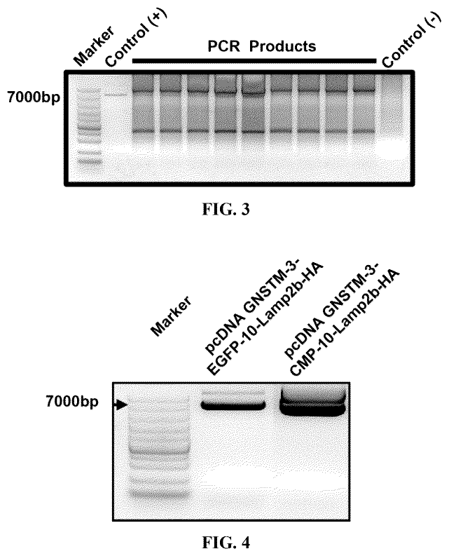

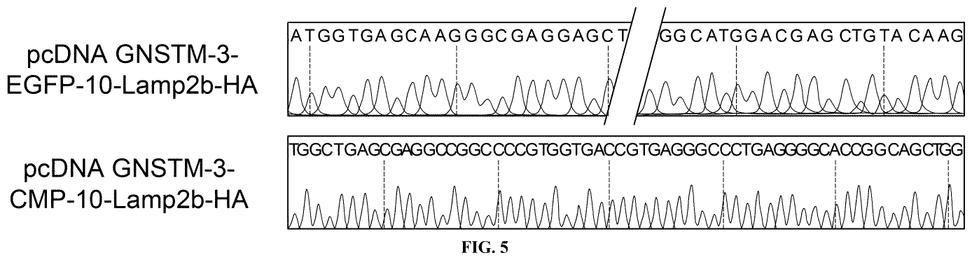

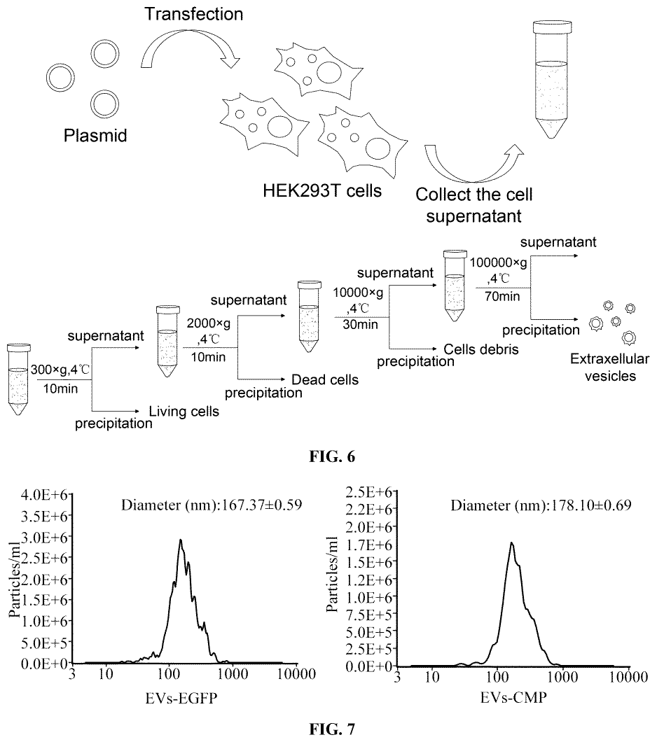

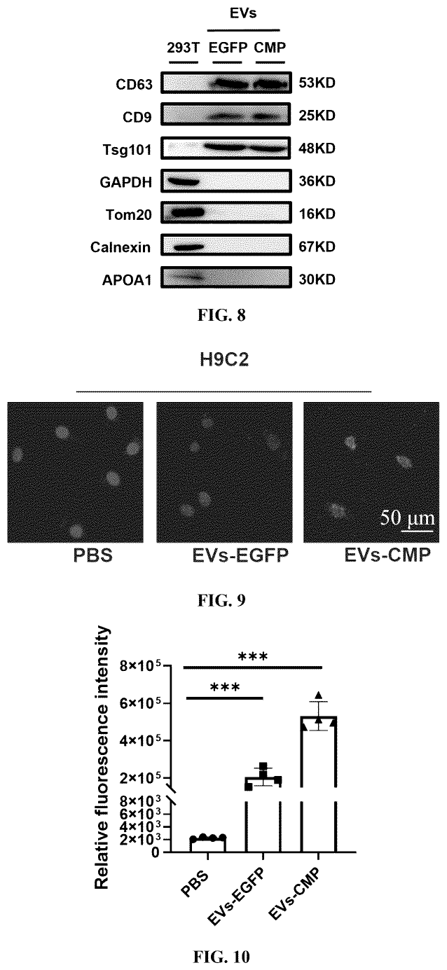

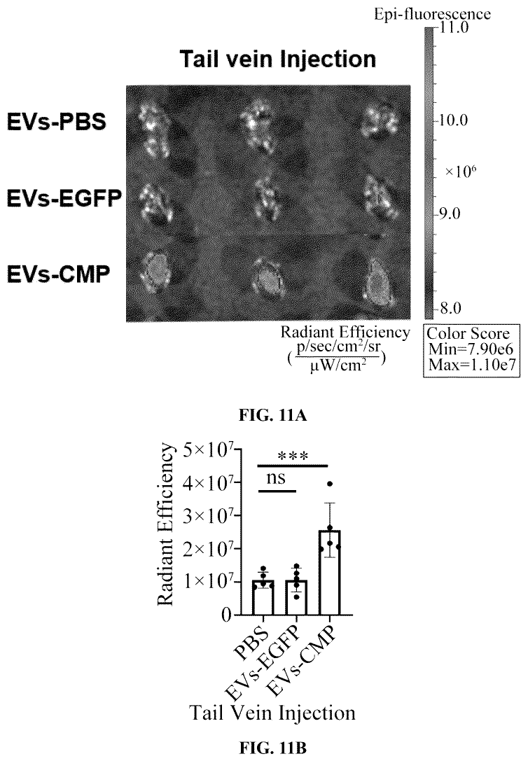

In the present disclosure, the fusion plasmid was constructed by fusing the nucleic acid sequence of peptide targeting cardiacmyocytes, CMP, with the nucleic acid sequence of Lamp-2b using homologous recombination, resulting in the generation of the fusion plasmid. The homologous recombination method involved the recombination of a linearized vector with the target sequence, yielding the fusion plasmid. A schematic representation of the homologous recombination method is shown in FIG. 1.

During homologous recombination, it is essential that the sequences at both ends of the target sequence and the linearized vector are identical, serving as homologous arms. Accordingly, sequences corresponding to the ends of the plasmid vector were designed at the 5′ and 3′ ends of the nucleic acid sequence of peptide targeting cardiacmyocytes, CMP. The 5′ end sequence was designed as follows: TGGGCAGTGGATACACCATT (SEQ ID NO: 1), and the 3′ end sequence was designed as: AATAGCAGAGGGAAGAGAGC (SEQ ID NO: 2). Additionally, an ATG sequence was appended after the 5′ homologous arm, and a GGAGGA sequence was added before the 3′ homologous arm. Therefore, the final design template for the synthesized sequence was: 5′-TGGGCAGTGGATACACCATTATG (SEQ ID NO: 3)-target sequence-GGAGGAAATAGCAGAGGGAAGAGAGC (SEQ ID NO: 4)-3′.

In the present disclosure, the synthesized nucleic acid sequence of the peptide targeting cardiacmyocytes, CMP, during the construction of the fusion plasmid was as follows: CMP-F: TGGGCAGTGGATACACCATTATGTGGCTGAGCGAGGCCGGCCCCGTGGTGACCGTGA GGGCCCTGAGGGGCACCGGCAGCTGGGGAGGAAATAGCAGAGGGAAGAGAGC (SEQ ID NO: 5);

CMP-R:
(SEQ ID NO: 6)
GCTCTCTTCCCTCTGCTATTTCCTCCCCAGCTGCCGGTGCCCCTCAGGGC
CCTCACGGTCACCACGGGGCCGGCCTCGCTCAGCCACATAATGGTGTATC
CACTGCCCA.

Furthermore, an enhanced green fluorescent protein (EGFP) was also modified onto the surface of the EVs as a negative control. Consequently, the nucleic acid sequence of EGFP was similarly fused with the nucleic acid sequence of Lamp-2b through homologous recombination to construct the fusion plasmid (EGFP plasmid). The nucleic acid sequence of EGFP was as follows:

(SEQ ID NO: 7)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAG.

The target sequences described in the present disclosure (i.e., the nucleic acid sequences of CMP and EGFP) were synthesized using conventional methods known in the field. Following annealing, the synthesized sequences were subjected to recombinant ligation.

The carrier plasmid for the fusion plasmid described in the present disclosure was the pcDNA GNSTM-3-RVG-10-Lamp2b-HA plasmid, with the plasmid map shown in FIG. 2. Primers were designed at both ends of the RVG sequence in order to linearize the plasmid vector using the PCR amplification method. The primers designed for the linearization of the plasmid vector were as follows:

PCR-F:
(SEQ ID NO: 8)
AATAGCAGAGGGAAGAGAGCATCC;
PCR-R:
(SEQ ID NO: 9)
AATGGTGTATCCACTGCCCATAG.

The linearized products obtained from the PCR were subjected to preliminary identification through agarose gel electrophoresis. In this analysis, Control (+) represents the positive control, which contains a correctly sequenced target fragment of approximately 7000 bp, while Control (−) represents the negative control, which does not contain any target fragment DNA. Subsequently, a gel recovery kit was used to recover the correctly identified products from the agarose gel (see FIG. 3), resulting in the linear pcDNA GNSTM-3-Lamp2b-HA plasmid. The results shown in FIG. 3 indicate that Lamp2b was successfully synthesized in the present disclosure. Sequencing confirmed that the nucleotide sequence of Lamp2b was as follows:

(SEQ ID NO: 10)
ATGGTGTGCTTCCGCCTCTTCCCGGTTCCGGGCTCAGGGCTCGTTCTGGT
CTGCCTAGTCCTGGGAGCTGTGCGGTCTTATGCATTGGAACTTAATTTGA
CAGATTCAGAAAATGCCACTTGCCTTTATGCAAAATGGCAGATGAATTTC
ACAGTACGCTATGAAACTACAAATAAAACTTATAAAACTGTAACCATTTC
AGACCATGGCACTGTGACATATAATGGAAGCATTTGTGGGGATGATCAGA
ATGGTCCCAAAATAGCAGTGCAGTTCGGACCTGGCTTTTCCTGGATTGCG
AATTTTACCAAGGCAGCATCTACTTATTCAATTGACAGCGTCTCATTTTC
CTACAACACTGGTGATAACACAACATTTCCTGATGCTGAAGATAAAGGAA
TTCTTACTGTTGATGAACTTTTGGCCATCAGAATTCCATTGAATGACCTT
TTTAGATGCAATAGTTTATCAACTTTGGAAAAGAATGATGTTGTCCAACA
CTACTGGGATGTTCTTGTACAAGCTTTTGTCCAAAATGGCACAGTGAGCA
CAAATGAGTTCCTGTGTGATAAAGACAAAACTTCAACAGTGGCACCCACC
ATACACACCACTGTGCCATCTCCTACTACAACACCTACTCCAAAGGAAAA
ACCAGAAGCTGGAACCTATTCAGTTAATAATGGCAATGATACTTGTCTGC
TGGCTACCATGGGGCTGCAGCTGAACATCACTCAGGATAAGGTTGCTTCA
GTTATTAACATCAACCCCAATACAACTCACTCCACAGGCAGCTGCCGTTC
TCACACTGCTCTACTTAGACTCAATAGCAGCACCATTAAGTATCTAGACT
TTGTCTTTGCTGTGAAAAATGAAAACCGATTTTATCTGAAGGAAGTGAAC
ATCAGCATGTATTTGGTTAATGGCTCCGTTTTCAGCATTGCAAATAACAA
TCTCAGCTACTGGGATGCCCCCCTGGGAAGTTCTTATATGTGCAACAAAG
AGCAGACTGTTTCAGTGTCTGGAGCATTTCAGATAAATACCTTTGATCTA
AGGGTTCAGCCTTTCAATGTGACACAAGGAAAGTATTCTACAGCTGAAGA
ATGTTCTGCTGACTCTGACCTCAACTTTCTTATTCCTGTTGCAGTGGGTG
TGGCCTTGGGCTTCCTTATAATTGTTGTCTTTATCTCTTATATGATTGGA
AGAAGGAAAAGTCGTACTGGTTATCAGTCTGTGTAA.

The nucleic acid sequence of the peptide targeting cardiacmyocytes, CMP, as set forth in SEQ ID NO: 5, was annealed with the nucleic acid sequence of EGFP, as set forth in SEQ ID NO: 8, according to the following procedure to obtain the desired insert fragment (i.e., the nucleic acid sequence of the peptide targeting cardiacmyocytes, CMP, or the nucleic acid sequence of EGFP): Reagents were added to a 1.5 mL centrifuge tube according to the annealing system described in Table 1. The tube was then placed in a water bath at 94° C. for 5 minutes. After the heating, the water bath was turned off, and the mixture was allowed to cool naturally to room temperature. Upon completion of the annealing process, the annealed product was stored at −20° C. for future use. The annealing system is detailed as follows:

TABLE 1
Annealing System

| Reagent | Amount | |
|---|---|---|
| Forward target sequence (10 nM) | 5 | μL |
| Reverse target sequence (10 nM) | 5 | μL |
| 10 × NEB Buffer | 5 | μL |
| Ultra-pure water | 35 | μL |

The forward primer sequence F:
(SEQ ID NO: 14)
5′-TGGGCAGTGGATACACCATTATG-3′
and
the reverse primer sequence R:
(SEQ ID NO: 15)
5′-GGAGGAAATAGCAGAGGGAAGAGAGC-3′
were utilized.

The reagents were added to a 1.5 mL centrifuge tube according to the homologous recombination reaction system described in Table 2, and mixing was performed by pipetting. The homologous recombination reaction system was prepared on ice, with the inserted target fragments being the EGFP nucleic acid sequence as set forth in SEQ ID NO: 8 or the CMP nucleic acid sequence as set forth in SEQ ID NO: 5.

TABLE 2
Homologous recombination reaction system

| Reagent | Amount | |
|---|---|---|
| Linearized vector fragment (linearized pcDNA | 3 | μL |
| GNSTM-3-Lamp2b-HA plasmid) | | |
| Inserted target fragment | 1 | μL |
| 5 × CE Buffer | 4 | μL |
| Exnase II | 2 | μL |
| Ultra-pure water | 10 | μL |

The homologous recombination reaction system was placed in a metal bath at 37° C. for 30 minutes, followed by incubation on ice or storage at low temperatures. After the completion of the homologous recombination reaction, the resulting recombinant products were subjected to transformation, monoclonal selection, and plasmid extraction. Subsequently, the plasmids were preliminarily identified through agarose gel electrophoresis (see FIG. 4), followed by Sanger sequencing, with the sequencing results presented in FIG. 5.

The results shown in FIGS. 4-5 indicate that a plasmid containing the fusion of the peptide targeting cardiacmyocytes, CMP, nucleic acid sequence with the Lamp-2b nucleic acid sequence (i.e., pcDNA GNSTM-3-CMP-10-Lamp2b-HA plasmid) was successfully constructed. Furthermore, a plasmid containing the fusion of the EGFP nucleic acid sequence with the Lamp-2b nucleic acid sequence (i.e., pcDNA GNSTM-3-EGFP-10-Lamp2b-HA plasmid) was also successfully constructed. The fusion sequence of the peptide targeting cardiacmyocytes, CMP, nucleic acid sequence and the Lamp-2b nucleic acid sequence was as follows: ATGGTGTGCTTCCGCCTCTTCCCGGTTCCGGGCTCAGGGCTCGTTCTGGTCTGCCTAG TCCTGGGAGCTGTGCGGTCTTATGCATTGGAACTTAATTTGACAGATTCAGAAAATGC CACTTGCCTTTATGCAAAATGGCAGATGAATTTCACAGTACGCTATGAAACTACAAAT AAAACTTATAAAACTGTAACCATTTCAGACCATGGCACTGTGACATATAATGGAAGCA TTTGTGGGGATGATCAGAATGGTCCCAAAATAGCAGTGCAGTTCGGACCTGGCTTTTC CTGGATTGCGAATTTTACCAAGGCAGCATCTACTTATTCAATTGACAGCGTCTCATTTT CCTACAACACTGGTGATAACACAACATTTCCTGATGCTGAAGATAAAGGAATTCTTAC TGTTGATGAACTTTTGGCCATCAGAATTCCATTGAATGACCTTTTTAGATGCAATAGTT TATCAACTTTGGAAAAGAATGATGTTGTCCAACACTACTGGGATGTTCTTGTACAAGC TTTTGTCCAAAATGGCACAGTGAGCACAAATGAGTTCCTGTGTGATAAAGACAAAAC TTCAACAGTGGCACCCACCATACACACCACTGTGCCATCTCCTACTACAACACCTACT CCAAAGGAAAAACCAGAAGCTGGAACCTATTCAGTTAATAATGGCAATGATACTTGTC TGCTGGCTACCATGGGGCTGCAGCTGAACATCACTCAGGATAAGGTTGCTTCAGTTAT TAACATCAACCCCAATACAACTCACTCCACAGGCAGCTGCCGTTCTCACACTGCTCTA CTTAGACTCAATAGCAGCACCATTAAGTATCTAGACTTTGTCTTTGCTGTGAAAAATG AAAACCGATTTTATCTGAAGGAAGTGAACATCAGCATGTATTTGGTTAATGGCTCCGT TTTCAGCATTGCAAATAACAATCTCAGCTACTGGGATGCCCCCCTGGGAAGTTCTTATA TGTGCAACAAAGAGCAGACTGTTTCAGTGTCTGGAGCATTTCAGATAAATACCTTTGA TCTAAGGGTTCAGCCTTTCAATGTGACACAAGGAAAGTATTCTACAGCTGAAGAATG TTCTGCTGACTCTGACCTCAACTTTCTTATTCCTGTTGCAGTGGGTGTGGCCTTGGGC TTCCTTATAATTGTTGTCTTTATCTCTTATATGATTGGAAGAAGGAAAAGTCGTACTGG TTATCAGTCTGTGTAATGGCTGAGCGAGGCCGGCCCCGTGGTGACCGTGAGGGCCCT GAGGGGCACCGGCAGCTGG (SEQ ID NO: 11). The peptide targeting cardiacmyocytes, CMP, nucleic acid sequence was as follows:

(SEQ ID NO: 12)
TGGCTGAGCGAGGCCGGCCCCGTGGTGACCGTGAGGGCCCTGAGGGGCAC
CGGCAGCTGG;

The amino acid sequence of the cardiac-targeting CMP was:

(SEQ ID NO: 13)
WLSEAGPVVTVRALRGTGSW.

2. Packaging, purification, and characterization of EVs

HEK293T cells were seeded in 10 cm cell culture dishes. When the cell density reached 3 million cells per dish, every 20 dishes were grouped together, and 10 μg of the target plasmid (pcDNA GNSTM-3-CMP-10-Lamp2b-HA or pcDNA GNSTM-3-EGFP-10-Lamp2b-HA) or 10 μg of PBS was added to each dish. Transfection was facilitated using 1 mg/ml polyethyleneimine (PEI). After 12 hours, the medium was replaced with high glucose DMEM containing 10% FBS, which did not contain EVs (the preparation of 10% FBS without EVs involved ultracentrifugation at 100,000×g for 10 hours at 4° C. to collect the supernatant, with strict sterile techniques maintained throughout). The cells were further cultured for 48 hours, followed by centrifugation at 300× g for 10 minutes to collect supernatant a. Supernatant a was subjected to centrifugation at 300×g for 10 minutes at 4° C. to remove viable cells, resulting in supernatant b. To eliminate dead cells, supernatant b was transferred to a new centrifuge tube and centrifuged at 2,000×g for 10 minutes at 4° C. to obtain supernatant c. Supernatant c was then subjected to centrifugation at 10,000×g for 30 minutes at 4° C. to remove cell debris, yielding supernatant d. Supernatant d was ultracentrifuged at 100,000×g for 70 minutes at 4° C. to obtain a pellet, which represented the cardiac-targeting EVs (denoted as EVs-CMP), EGFP-modified EVs (denoted as EVs-EGFP), and control EVs (denoted as PBS, purchased from Yuanzi Medical, which does not contain EVs). The supernatant was discarded, and the EVs were resuspended in 1×PBS and transferred to a 1.5 mL centrifuge tube. The preparation workflow for the cardiac-targeting EVs is illustrated in FIG. 6.

Measurement of EV size and particle number: the size and particle number of EVs-CMP and EVs-EGFP were measured using a nanoparticle tracking analyzer (Particle Metrix). Initially, the instrument was cleaned with grade I ultrapure water filtered with 0.22 μm filter and 1×PBS. Subsequently, polystyrene (PS) beads standard were diluted 250,000-fold for calibration. After calibration, the instrument was cleaned again with 1×PBS, and the samples to be measured were diluted 5,000-fold before measurement. Thus, the size and particle number of the EVs were obtained (see FIG. 7).

The results in FIG. 7 indicate that the size of EVs-CMP was measured to be 167.37±0.59 nm, while the size of EVs-EGFP was measured to be 178.10±0.69 nm.

Detection of EV surface marker proteins: the expression of EV surface marker proteins CD63, CD9, Tsg101, GAPDH, Tom20, Calnexin, and APOA1 in HEK293T cells, EVs-CMP, and EVs-EGFP was assessed using Western blotting.

The results demonstrated that CD63, CD9, and Tsg101 were expressed in both EVs-CMP and EVs-EGFP, while GAPDH, Tom20, Calnexin, and APOA1 were only expressed in HEK293T cells, serving as negative controls (see FIG. 8).

The results presented in FIGS. 7-8 indicate that successful construction of cardiac-targeting EVs and EGFP-modified EVs was achieved.

### Example 2

Investigation of the cardiac targeting function of EVs modified with peptide targeting cardiacmyocytes, CMP, prepared in Example 1.

### 1. Fluorescent Dye Labeling of EVs:

the EVs-CMP were labeled with Did fluorescent dye. The EVs were diluted in 1×PBS to a volume of 1 mL, and 10 μL of 1 μg/μL Did dye was added to the centrifuge tube. The mixture was incubated at room temperature in the dark for 30 minutes. To remove free dye, ultracentrifugation was performed at 100,000×g for 70 minutes at 4° C. The supernatant was discarded, and the pellet at the bottom of the ultracentrifuge tube was resuspended in 200 μL of 1×PBS to obtain the Did-labeled EVs-CMP.

### 2. Cell-Level Validation of Cardiac Targeting of EVs:

H9C2 cells, HepG2 cells, BEAS-2B cells, and HEK293T cells were treated with PBS, the prepared Did-stained EVs-CMP, or EVs-EGFP, with a quantity of 1×10¹⁰ particles/mL added to each well (50 μL per well). After a 3-hour incubation, the culture medium was removed, and the cell nuclei were stained using Hoechst 33342. Confocal microscopy was employed to capture images and observe the uptake of EVs by the cells, with HepG2 cells, BEAS-2B cells, and HEK293T cells serving as control groups. The results are presented in FIGS. 9-10.

Furthermore, flow cytometry was utilized to assess the uptake of EVs by H9C2 cells, with results shown in FIG. 10.

The findings from FIGS. 9-10 indicate that, in comparison to PBS, HepG2 cells, BEAS-2B cells, HEK293T cells, and EVs-EGFP, EVs-CMP were effectively taken up by H9C2 cells.

### 3. Animal-Level Validation of Cardiac Targeting of EVs:

Tail vein injection was performed to administer the fluorescently labeled EVs-CMP, EVs-EGFP, and EVs-PBS into mice (200 μL per mouse, 1×10⁸ particles/μL). After 24 hours, the hearts of the mice were excised for fluorescence imaging.

In vivo imaging results demonstrated that EVs-CMP could accumulate in the mouse heart (see FIG. 11A-FIG. 11B).

The results indicate that EVs modified with CMP exhibited good targeting ability at both the cellular and animal levels. Consequently, EVs-CMP represents a promising novel tool for cardiac-targeting delivery.

### Example 3

1. Construction of Stable miR-30d-Expressing Cell Line

The method for constructing a stable miR-30d-expressing cell line was as follows.

(1) HEK293T cells were seeded in a 10 cm cell culture dish and cultured in a 5% CO2, 37° C. environment until the cell density reached approximately 95% confluence before transfection. Prior to transfection, a transfection solution was prepared by sequentially adding 1.25 μg pMD2-G, 3.75 μg psPAX.2G, and 5 μg of the miR-30d-OE target plasmid to serum-free DMEM, followed by the addition of 100 μg of 1 mg/mL PEI. The mixture was thoroughly vortexed and allowed to stand for 15 minutes. Then, 1 mL of the mixture was evenly added to each dish, and the dishes were gently swirled. The miR-30d-OE target plasmid was constructed by retrieving the sequence of miR-30d (UGUAAACAUCCCCGACUGGAAG, SEQ ID NO: 16) from the miRDB website. The U in the sequence was replaced with T to obtain the new sequence (TGTAAACATCCCCGACTGGAAG, SEQ ID NO: 17). This sequence was then inserted in reverse order into the pLKO.1-TRC vector (purchased from Addgene: #10878), and the miR-30d-OE target plasmid was synthesized by Tsingke Biotechnology Co., Ltd.

After 10 to 12 hours of transfection, the complete culture medium was replaced.

(2) After 48 hours of culture, the cell supernatant was collected into a 50 mL centrifuge tube.

(3) The supernatant was collected again after an additional 24 hours of culture into another 50 mL centrifuge tube.

(4) The collected lentivirus in the supernatant was filtered using a 0.22 μm filter and stored at −20° C.

(5) Prior to constructing the stable-transfected cell line, HEK293T cells were cultured until the cell density reached approximately 70%.

(6) The pre-packaged lentivirus was mixed with serum-free culture medium in a 1:1 volume ratio and added to the culture dish. Polybrene was included to enhance the lentivirus transfection efficiency before the mixing.

(7) After 12 hours of adding the virus, the culture medium was replaced with complete medium.

(8) After 48 hours of medium replacement, to allow the virus to express within the cells for some time, the cells were passaged. During passaging, an appropriate amount of puromycin was added to the medium to kill uninfected HEK293T cells. This passaging method was repeated 2-3 times until nearly all cells in the control group had died, at which point the treatment was stopped.

(9) Subsequently, the surviving cells that had been treated with the lentivirus and were not killed by puromycin were cultured in complete medium.

(10) Real-time quantitative RT-PCR was performed to detect the expression level of miR-30d-OE in the cell line, confirming whether the stable-transfected cell line had been successfully constructed.

(11) When the density of HEK293T cells reached over 90%, the fusion plasmid (CMP plasmid) was transfected into the culture dish at a dosage of 10 μg per well.

(12) After 12 hours of transfection, fresh complete medium was replaced (the serum used in this medium had to be pre-treated to remove EVs by ultra-centrifugation at 100,000×g for 10 hours at 4° C., ensuring strict sterile conditions throughout the process).

(13) After 24 hours of medium replacement, the cell supernatant containing EVs was collected.

(14) The cell supernatant was centrifuged at 300×g for 10 minutes at 4° C., and the supernatant was retained while the pellet was discarded.

(15) The cell supernatant was then centrifuged at 2000×g for 10 minutes at 4° C., retaining the supernatant and discarding the pellet.

(16) The cell supernatant underwent centrifugation at 10,000×g for 30 minutes at 4° C., with the supernatant retained and the pellet discarded.

(17) The supernatant was filtered using a 0.22 μm filter, and after filtration, the next step of ultra-centrifugation was performed.

(18) The cell supernatant was subjected to ultra-centrifugation at 100,000×g for 70 minutes at 4° C., after which the supernatant was discarded. The pellet at the bottom of the ultra-centrifuge tube was resuspended in 1 mL of PBS (1×), resulting in a suspension that contained EVs, designated as EVs-CMP-miR-30d.

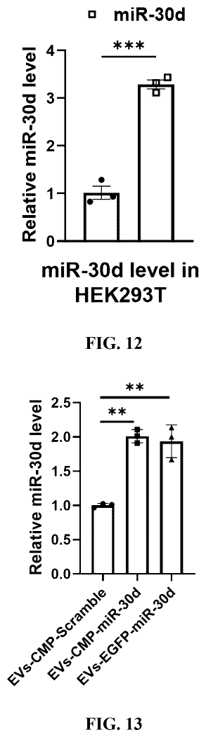

Initially, a plasmid capable of overexpressing miR-30d (pLKO.1-miR-30d) was constructed, and this plasmid was used to package lentivirus, which was then employed to establish a stable miR-30d-expressing cell line (referred to as miR-30d HEK293T cells). Real-time quantitative RT-PCR was conducted to measure the level of miR-30d in the stable miR-30d-transfected HEK293T cells, with untransfected HEK293T cells serving as the control group (referred to as Scramble). The results are shown in FIG. 12.

The data in FIG. 12 indicate that the level of miR-30d in the stable miR-30d HEK293T cells was significantly higher than that in the control group, confirming the successful establishment of the stable miR-30d HEK293T cell line.

2. Packaging, Purification, and Detection of miR-30d-Loaded Cardiac Targeting EVs

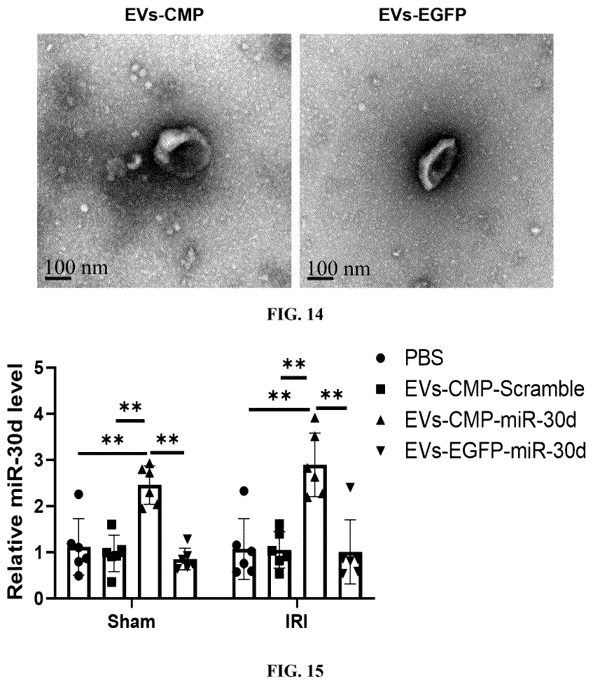

Stable miR-30d-expressing cell lines were cultured to an appropriate density for the production of miR-30d-loaded EVs. The specific experimental groups were as follows: Group 1: EVs packaged from a stable-transfected cell line constructed with control lentivirus (EVs-CMP-Scramble); Group 2: EVs produced from a stable cell line overexpressing miR-30d and modified on the surface with peptide targeting cardiacmyocytes, CMP, (EVs-CMP-miR-30d); Group 3: EVs packaged from a stable cell line overexpressing miR-30d and modified on the surface with EGFP (EVs-EGFP-miR-30d). The preparation of EVs-CMP-Scramble differed from the aforementioned “1” section in that for step (1), 5 μg of scramble control plasmid was added, while the remaining steps were consistent with those described in section “1.” The sequence of the scramble was GGCGGGTTAATCATTAACTACAAGGAACCC (SEQ ID NO: 18), and the construction method of the scramble plasmid is referenced in Example 1 regarding the fusion plasmid (EGFP plasmid). The preparation of EVs-EGFP-miR-30d differed from the aforementioned “1” section in that in step (11), 10 μg of the fusion plasmid (EGFP) from Example 1 was transfected into each well of the cell culture dish, while the remaining steps were consistent with those described in section “1”. The levels of miR-30d within the EVs-CMP-Scramble, EVs-CMP-miR-30d, and EVs-EGFP-miR-30d were detected using qRT-PCR. The results indicated that the levels of miR-30d in the EVs-CMP-miR-30d and EVs-EGFP-miR-30d were significantly elevated compared to EVs-CMP-Scramble (see FIG. 13). Transmission electron microscopy (TEM) was utilized to assess the size and morphology of the EVs-CMP-miR-30d and EVs-EGFP-miR-30d. The results from FIG. 14 demonstrated that the size and morphology of the miR-30d-loaded cardiac-targeting EVs were consistent with expectations. FIGS. 12-14 collectively indicate that a novel miR-30d-loaded cardiac-targeting EV was constructed in this study.

### Example 4

Effects of miR-30d-Loaded Cardiac-Targeting EVs (EVs-CMP-miR-30d) on Heart Failure Induced by Acute Ischemia-Reperfusion Injury

### 1. Injection of EVs Via Tail Vein in Mice

EVs, specifically EVs-CMP-Scramble, EVs-CMP-miR-30d, and EVs-EGFP-miR-30d, prepared in Example 3, were injected into mice via the tail vein. A dose of 2× 1010 particles per mouse was administered directly. The acute ischemia-reperfusion injury model was established 12 hours post-injection.

### 2. Establishment of the Acute Ischemia-Reperfusion Injury (IRI) Model in Mice

Mice were anesthetized using 4% chloral hydrate. The neck skin, muscle, and adipose tissue were first separated, and the trachea was exposed. A surgical scissors was used to cut the cartilage rings of the trachea, allowing for the insertion of a tracheal cannula, which was then secured. The ventilator was set to a frequency of 120 breaths per minute with a tidal volume of 2 mL. Following verification of normal chest contour and respiration, subsequent surgeries were performed. A transverse thoracotomy was conducted between the fourth and fifth left ribs, with an incision of approximately 1 cm. The skin and thoracic muscle were dissected layer by layer using surgical scissors. The intercostal muscle was bluntly dissected to expose the heart, and a 4-0 surgical suture was used to ligate the left anterior descending artery (success was indicated by blanching of the apex post-ligation). Hemostatic clamps were applied to the incised skin, and the chest was temporarily closed for 30 minutes of ischemia. After 30 minutes, the ligature used for the heart was cut. The intercostal muscle, thoracic muscle, and skin were sutured layer by layer. The tracheal cannula was removed, and the postoperative wound was disinfected with iodine. Mice were placed on a heating pad to allow for spontaneous breathing until consciousness was regained and they could crawl independently, at which point they were returned to their cages. The sham surgery group underwent identical procedures except for the 30 minutes of left anterior descending artery ligation. The specific grouping was as follows: Mice were treated using a 1 mL sterile syringe via tail vein injection, with the experimental groups divided into eight groups.

Group 1: PBS+sham surgery group, injected with 200 μL of PBS per mouse, followed by sham surgery 12 hours post-injection.

Group 2: EVs-CMP-Scramble+sham surgery group, injected with 200 μL of EVs-CMP-Scramble per mouse, followed by sham surgery 12 hours later.

Group 3: EVs-CMP-miR-30d+sham surgery group, injected with 200 μL of EVs-CMP-miR-30d per mouse, followed by sham surgery 12 hours later.

Group 4: EVs-EGFP-miR-30d+sham surgery group, injected with 200 μL of EVs-EGFP-miR-30d per mouse, followed by sham surgery 12 hours later.

Group 5: PBS+surgery group, injected with 200 μL of PBS per mouse, followed by acute ischemia-reperfusion surgery 12 hours later.

Group 6: EVs-CMP-Scramble+surgery group, injected with 200 μL of EVs-CMP-Scramble per mouse, followed by acute ischemia-reperfusion surgery 12 hours later.

Group 7: EVs-CMP-miR-30d+surgery group, injected with 200 μL of EVs-CMP-miR-30d per mouse, followed by acute ischemia-reperfusion surgery 12 hours later.

Group 8: EVs-EGFP-miR-30d+surgery group, injected with 200 μL of EVs-EGFP-miR-30d per mouse, followed by acute ischemia-reperfusion surgery 12 hours later.

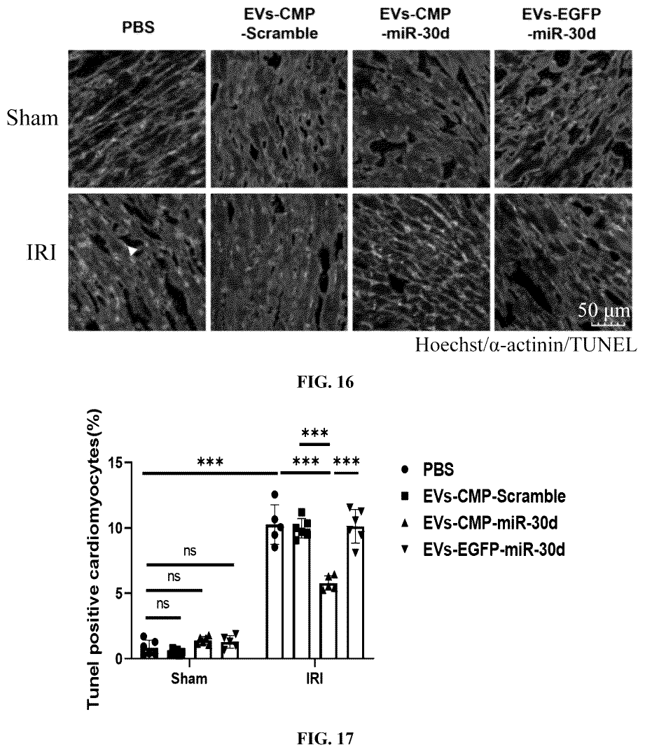

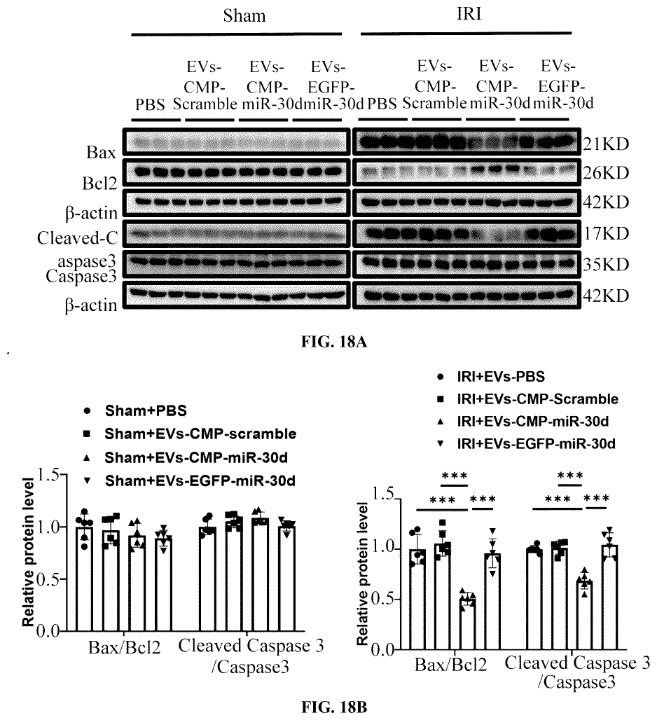

Twenty-four hours post-injection, the experiment was concluded, and the hearts of the mice were dissected. Tissue samples were collected to extract total RNA from heart tissues, and qRT-PCR was employed to detect changes in miR-30d levels. Additionally, immunofluorescence staining was conducted to assess the rate of cardiomyocyte apoptosis, and the apoptosis of cardiomyocytes was quantified. Total protein was also extracted from the tissues, and Western blotting was performed to analyze changes in apoptosis-related proteins. The results indicated that the injection of EVs-CMP-miR-30d led to an increase in the levels of miR-30d in heart tissues (FIG. 15). Immunofluorescence staining results showed that cardiomyocyte apoptosis occurred in the acute ischemia-reperfusion injury model; however, the injection of EVs-CMP-miR-30d provided protection against cardiomyocyte apoptosis (see FIGS. 16-17). The Western blotting results demonstrated that the levels of apoptosis-related proteins Bax/Bcl2 and Caspase3 were alleviated following the injection of EVs-CMP-miR-30d (FIG. 18A-FIG. 18B). Therefore, it was concluded that miR-30d-loaded cardiac-targeting EVs (EVs-CMP-miR-30d) could protect against heart failure induced by acute ischemia-reperfusion injury.

### Example 5

Effects of miR-30d-Loaded Cardiac-Targeting EVs (EVs-CMP-miR-30d) on Ventricular Remodeling Induced by Ischemia-Reperfusion Injury
1. Establishment of a Ventricular Remodeling Model Induced by Ischemia-Reperfusion Injury in Mice after 3 Weeks

The acute ischemia-reperfusion injury surgery in mice was performed as described in Example 4. Following the surgery, EV injections were administered to the mice at scheduled intervals (on Days 1, 2, 4, 6, 9, 12, 15, 18, and 21 post-surgery, with a dosage of 2× 10¹⁰ particles per mouse and an injection volume of 200 μL). The experiment concluded after 3 weeks post-surgery, and the hearts of the mice were harvested for dissection. Tissue samples were obtained, total RNA was extracted from the cardiac tissue, and the changes in miR-30d levels were detected using qRT-PCR. Cardiac function in the mice was assessed via echocardiography, and fibrosis levels in the heart were evaluated using Masson's trichrome staining. The EVs used included EVs-CMP-Scramble, EVs-CMP-miR-30d, and EVs-EGFP-miR-30d, which were prepared as described in Example 3. The experimental groups were divided as follows:

Group 1: PBS+sham surgery group, receiving 200 μL/mouse of PBS.

Group 2: EVs-CMP-Scramble+sham surgery group, receiving 200 μL/mouse of EVs-CMP-Scramble.

Group 3: EVs-CMP-miR-30d+sham surgery group, receiving 200 μL/mouse of EVs-CMP-miR-30d.

Group 4: EVs-EGFP-miR-30d+sham surgery group, receiving 200 μL/mouse of EVs-EGFP-miR-30d.

Group 5: PBS+surgery group, receiving 200 μL/mouse of PBS.

Group 6: EVs-CMP-Scramble+surgery group, receiving 200 μL/mouse of EVs-CMP-Scramble.

Group 7: EVs-CMP-miR-30d+surgery group, receiving 200 μL/mouse of EVs-CMP-miR-30d.

Group 8: EVs-EGFP-miR-30d+surgery group, receiving 200 μL/mouse of EVs-EGFP-miR-30d.

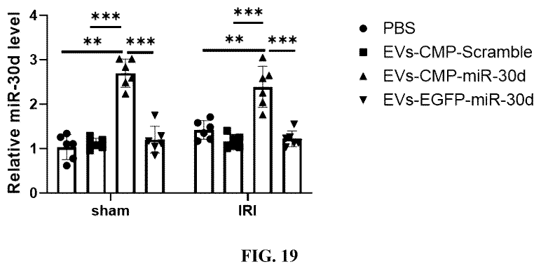

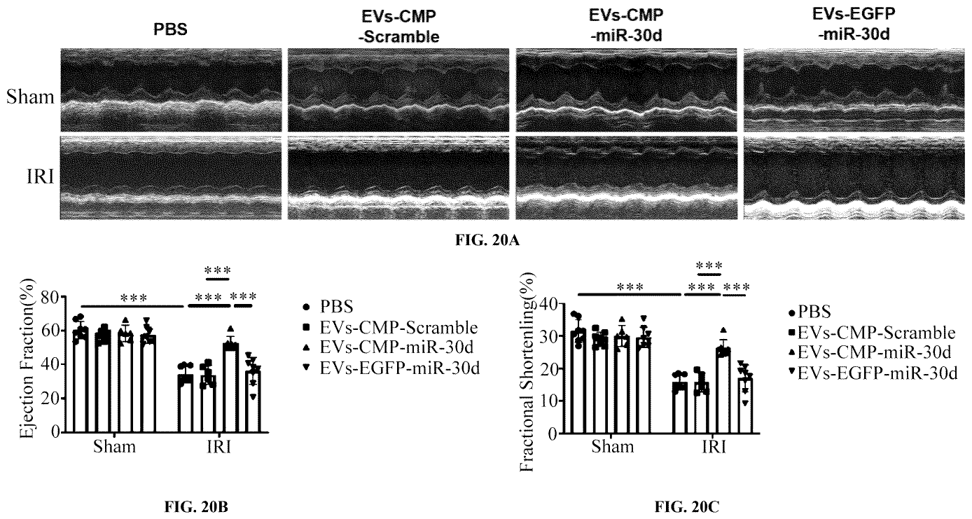

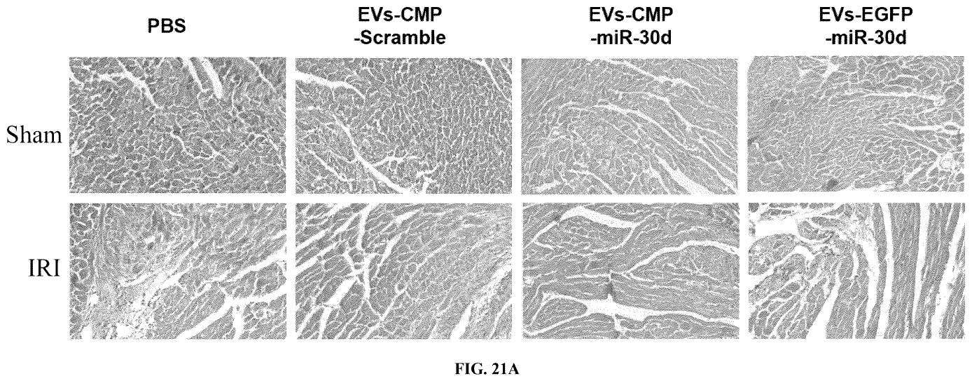

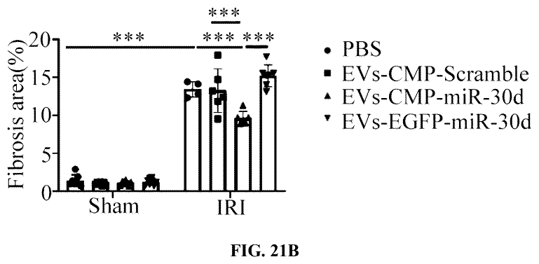

QRT-PCR was utilized to measure the levels of miR-30d in cardiac tissue following the injection of different EVs. Echocardiography was employed to assess cardiac function in the mice post-injection of various EVs. Masson's trichrome staining was performed to evaluate the fibrosis levels in the hearts of the mice after the injection of different EVs. The results indicated that in mice injected with EVs-CMP-miR-30d, the levels of miR-30d in cardiac tissue were significantly increased as demonstrated by qRT-PCR (see FIG. 19). Echocardiographic analysis revealed that cardiac function declined significantly three weeks after the ischemia-reperfusion injury surgery; however, injection of EVs-CMP-miR-30d was found to improve cardiac function in the mice (see FIG. 20A-FIG. 20C). Additionally, Masson's staining results showed that the fibrosis levels in the hearts of the mice were improved following the injection of EVs-CMP-miR-30d (see FIG. 21A-FIG. 21B). Collectively, these results suggest that EVs loaded with miR-30d can ameliorate ventricular remodeling induced by ischemia-reperfusion injury in mice.

### Example 6

Effects of miR-30d-Loaded Cardiac-Targeting EVs (EVs-CMP-miR-30d) on Myocardial Hypertrophy (TAC)

### 1. Establishment of Myocardial Hypertrophy Model Induced by Aortic Arch Constriction in Mice

Mice were anesthetized with 4% chloral hydrate. The neck skin, muscles, and adipose tissue were first dissected. After the trachea was exposed, surgical scissors were used to cut the cartilaginous rings of the organs, creating an opening suitable for tracheal intubation, which was then secured. The ventilator was set to a frequency of 120 breaths/min and a tidal volume of 2 mL. Following verification of normal chest contour and respiration in the mice, subsequent surgical procedures were performed. Next, two ribs were cut downward from the tracheal opening, and rib retractors were used to widen the rib cage. The skin and chest muscles were separated layer by layer to expose the aortic arch, which was constricted using 7-0 non-absorbable sutures. Subsequently, the intercostal muscles, chest muscles, and skin were sutured layer by layer. The tracheal tube was removed, and the surgical site was disinfected with iodine. Mice were transferred to a heating pad to allow for spontaneous respiration until consciousness was regained and the ability to crawl was restored, at which point they were returned to their cages. The sham-surgery group underwent similar procedures except for the aortic arch constriction.

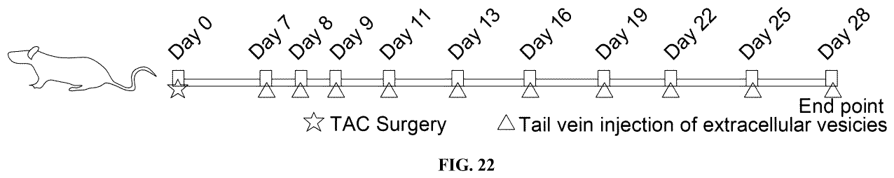

Following the aforementioned surgical procedures for myocardial hypertrophy, EV injections were administered at scheduled intervals (On Days 7, 8, 9, 11, 13, 16, 19, 22, 25, and 28 post-surgery, with a dosage of 2× 10¹⁰ particles/mouse and an injection volume of 200 μL). The experiment concluded five weeks post-surgery, and the mice hearts were dissected for analysis. Tissue samples were collected for total RNA extraction from cardiac tissues. The changes in miR-30d levels were assessed using qRT-PCR, while echocardiography was employed to evaluate cardiac function. Masson's staining was utilized to determine the level of cardiac fibrosis, and HE and WGA staining were performed to assess cardiomyocyte size. The EVs used were EVs-CMP-Scramble, EVs-CMP-miR-30d, and EVs-EGFP-miR-30d, prepared as described in Example 3. The experimental workflow for assessing the impact of EVs on myocardial hypertrophy is illustrated in FIG. 22. The specific groupings for the experiment were as follows:

Group 1: Sham-surgery+PBS, receiving 200 μL PBS per mouse.

Group 2: Sham-surgery+EVs-CMP-Scramble, receiving 200 μL EVs-CMP-Scramble per mouse.

Group 3: Sham-surgery+EVs-CMP-miR-30d, receiving 200 μL EVs-CMP-miR-30d per mouse.

Group 4: Sham-surgery+EVs-EGFP-miR-30d, receiving 200 μL EVs-EGFP-miR-30d per mouse.

Group 5: Operated+PBS, receiving 200 μL PBS per mouse.

Group 6: Operated+EVs-CMP-Scramble, receiving 200 μL EVs-CMP-Scramble per mouse.

Group 7: Operated+EVs-CMP-miR-30d, receiving 200 μL EVs-CMP-miR-30d per mouse.

Group 8: Operated+EVs-EGFP-miR-30d, receiving 200 μL EVs-EGFP-miR-30d per mouse.

QRT-PCR was conducted to measure the levels of miR-30d in cardiac tissues following the injection of different EVs. Echocardiography was used to evaluate cardiac function post-injection of various EVs. Masson's staining was performed to assess cardiac fibrosis levels, while HE and WGA staining were used to evaluate cardiomyocyte size.

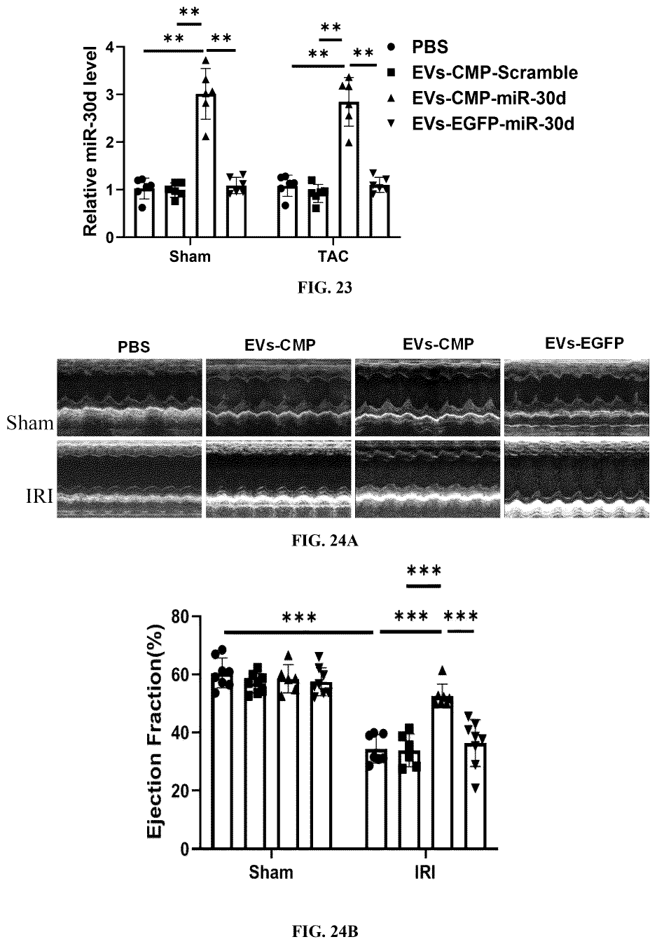

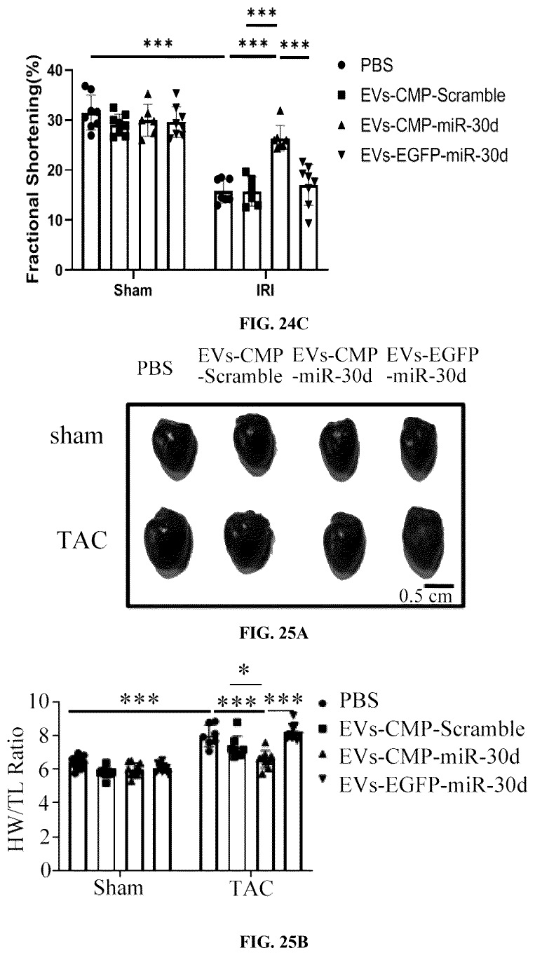

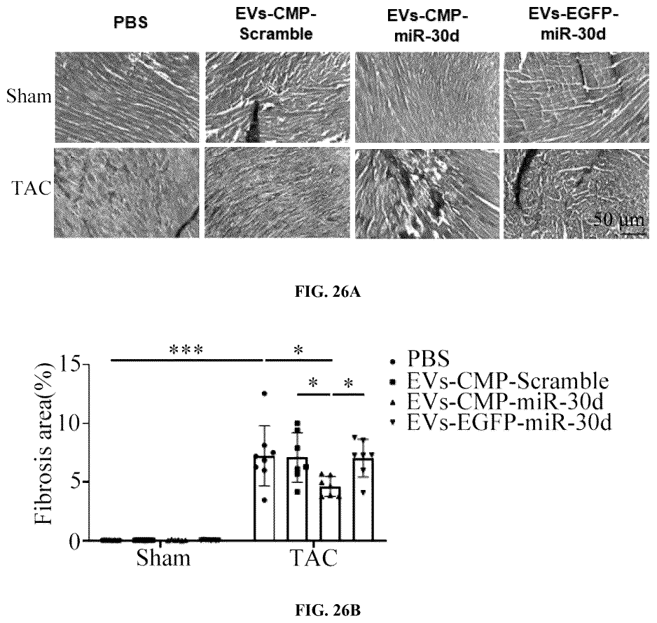

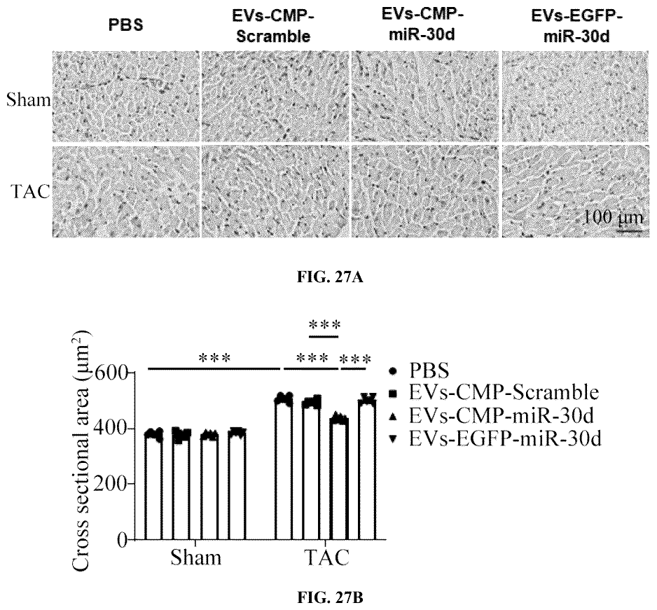

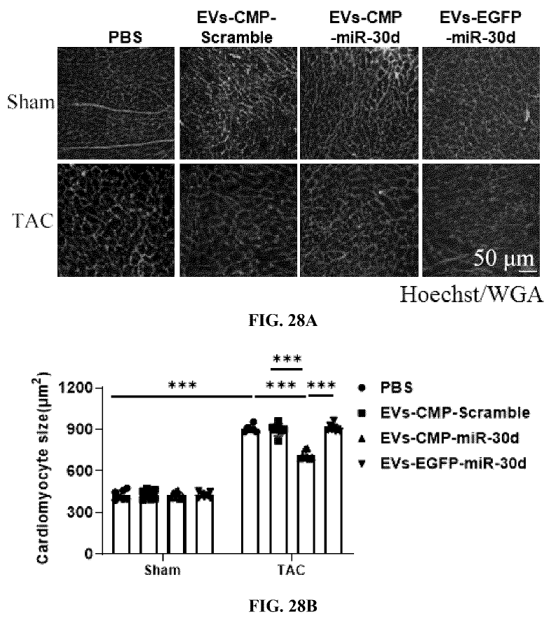

Results indicated that the injection of EVs-CMP-miR-30d led to an increase in miR-30d levels in cardiac tissues, as shown by real-time quantitative PCR (FIG. 23). Echocardiography results demonstrated that the injection of EVs-CMP-miR-30d improved cardiac function in mice subjected to the myocardial hypertrophy model (FIG. 24A-FIG. 24C), and cardiac size was reduced (FIG. 25A-FIG. 25B). Masson's staining revealed an improvement in cardiac fibrosis levels following the injection of EVs-CMP-miR-30d (FIG. 26A-FIG. 26B). HE and WGA staining showed that cardiomyocyte size was also improved post-injection of EVs-CMP-miR-30d (FIGS. 27A-27B and FIGS. 28A-28B). Collectively, these results suggest that miR-30d-loaded EVs can ameliorate myocardial hypertrophy induced by aortic arch constriction in mice.

The above description represents preferred embodiments of the present disclosure. It should be noted that persons of ordinary skill in this field may make several modifications and refinements without departing from the principles of the present disclosure, and such modifications and refinements should also be considered within the protection scope of the present disclosure.

What is claimed is:

1. A cardiac-targeting extracellular vesicle (EV), comprising a fusion sequence of lysosome-associated membrane protein 2, isoform b (Lamp-2b) and a cardiacmyocyte-specific peptide (CMP); wherein the fusion sequence of the Lamp-2b and the CMP is set forth in SEQ ID NO: 11.

2. A method for preparing the cardiac-targeting EV of claim 1, comprising
inserting a sequence of the CMP into a plasmid-cDNA (pcDNA) GNSTM-3-rabies virus glycoprotein (RVG)-10-Lamp2b-human influenza hemagglutinin (HA) plasmid to obtain a recombinant plasmid, transfecting the recombinant plasmid into a host cell to obtain a recombinant cell, and culturing the recombinant cell to secrete and isolate the cardiac-targeting EV,
wherein the host cell is a human embryonic kidney 293 cell expressing the SV40 large T antigen (HEK293T) cell.

3. The method of claim 2, wherein the nucleotide sequence of the CMP is set forth in SEQ ID NO: 12 and the amino acid sequence of the CMP is set forth in SEQ ID NO: 13.

4. The method of claim 2, wherein a ratio of the recombinant plasmid to the host cell is 8-12 μg: 2.8-3.2 million cells.

5. An EV for treating cardiovascular disease, wherein the EV for treating the cardiovascular disease is obtained by loading the cardiac-targeting EV of claim 1 with miR-30d.

6. A method for preparing the EV for treating the cardiovascular disease of claim 5, comprising
inserting a sequence of the CMP into a pcDNA GNSTM-3-RVG-10-Lamp2b-HA plasmid to obtain a recombinant plasmid, transfecting the recombinant plasmid into a host cell to obtain a recombinant cell, culturing the recombinant cell to secrete the EV, and isolating the EV for treating the cardiovascular disease, wherein
the host cell is a stable miR-30d-transfected HEK293T cell; and
the stable miR-30d-transfected HEK293T cell is prepared by co-transfecting a plasmid and a packaging plasmid into an HEK293T cell, wherein the plasmid overexpresses the miR-30d.

7. A method for treating cardiovascular disease, comprising administering to a subject in need thereof a therapeutic effect amount of the EV of claim 5.

8. The method of claim 7, wherein the EV is prepared by inserting a sequence of the CMP.

9. A method of targeted therapy for cardiovascular disease, comprising administering to a subject in need thereof a therapeutic effect amount of the EV of claim 5.

10. The method of claim 9, wherein the EV is prepared by inserting a sequence of the CMP into a pcDNA GNSTM-3-RVG-10-Lamp2b-HA plasmid to obtain a recombinant plasmid, transfecting the recombinant plasmid into a host cell to obtain a recombinant cell, culturing the recombinant cell to secrete the EV, and isolating the EV for treating the cardiovascular disease,
wherein the host cell is a stable miR-30d-transfected HEK293T cell; and
the stable miR-30d-transfected HEK293T cell is prepared by co-transfecting a plasmid and a packaging plasmid into an HEK293T cell, wherein the plasmid overexpresses the miR-30d.

11. A drug for treating cardiovascular disease, wherein the drug comprises the EV of claim 5, and a pharmaceutically acceptable excipient.

12. The drug of claim 11, wherein the EV is prepared by inserting a sequence of the CMP into a pcDNA GNSTM-3-RVG-10-Lamp2b-HA plasmid to obtain a recombinant plasmid, transfecting the recombinant plasmid into a host cell to obtain a recombinant cell, culturing the recombinant cell to secrete the EV, and isolating the EV for treating the cardiovascular disease,
wherein the host cell is a stable miR-30d-transfected HEK293T cell; and
the stable miR-30d-transfected HEK293T cell is prepared by co-transfecting a plasmid and a packaging plasmid into an HEK293T cell, wherein the plasmid overexpresses the miR-30d.


SEQUENCE LISTING (20 sequences)

SEQ ID NO: 1 - DNA, 20 bp, synthetic construct
  source 1..20: mol_type = other DNA; note = 5' end of the nucleic acid sequence of cardiac-targeting peptide CMP
tgggcagtggatacaccatt

SEQ ID NO: 2 - DNA, 20 bp, synthetic construct
  source 1..20: mol_type = other DNA; note = 3' end of the nucleic acid sequence of cardiac-targeting peptide CMP
aatagcagagggaagagagc

SEQ ID NO: 3 - DNA, 23 bp, synthetic construct
  source 1..23: mol_type = other DNA; note = Fragment of the final design template for the synthesized sequence
tgggcagtggatacaccattatg

SEQ ID NO: 4 - DNA, 26 bp, synthetic construct
  source 1..26: mol_type = other DNA; note = Fragment of the final design template for the synthesized sequence
ggaggaaatagcagagggaagagagc

SEQ ID NO: 5 - DNA, 109 bp, synthetic construct
  source 1..109: mol_type = other DNA; note = CMP-F
tgggcagtggatacaccattatgtggctgagcgaggccggccccgtggtgaccgtgagggccctgaggggcaccggcagctggggaggaaatagcagagggaagagagc

SEQ ID NO: 6 - DNA, 109 bp, synthetic construct
  source 1..109: mol_type = other DNA; note = CMP-R
gctctcttccctctgctatttcctccccagctgccggtgcccctcagggccctcacggtcaccacggggccggcctcgctcagccacataatggtgtatccactgccca

SEQ ID NO: 7 - DNA, 669 bp, synthetic construct
  source 1..669: mol_type = other DNA; note = Nucleic acid sequence of EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggag

SEQ ID NO: 8 - DNA, 24 bp, synthetic construct
  source 1..24: mol_type = other DNA; note = Primer PCR-F
aatagcagagggaagagagcatcc

SEQ ID NO: 9 - DNA, 23 bp, synthetic construct
  source 1..23: mol_type = other DNA; note = Primer PCR-F
aatggtgtatccactgcccatag

SEQ ID NO: 10 - DNA, 1236 bp, synthetic construct
  source 1..1236: mol_type = other DNA; note = Nucleotide sequence of Lamp2b
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaa

SEQ ID NO: 11 - DNA, 1296 bp, synthetic construct
  source 1..1296: mol_type = other DNA; note = Fusion sequence of Lamp-2b and cardiac-targeting peptide CMP
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaatggctgagcgaggccggccccgtggtgaccgtgagggccctgaggggcaccggcagctgg

SEQ ID NO: 12 - DNA, 60 bp, synthetic construct
  source 1..60: mol_type = other DNA; note = Nucleotide sequence encoding the cardiac-targeting peptide CMP
tggctgagcgaggccggccccgtggtgaccgtgagggccctgaggggcaccggcagctgg

SEQ ID NO: 13 - protein, 20 aa, synthetic construct
  source 1..20: note = Amino acid sequence of the cardiac-targeting peptide CMP
WLSEAGPVVTVRALRGTGSW

SEQ ID NO: 14 - DNA, 23 bp, synthetic construct
  source 1..23: mol_type = other DNA; note = Forward primer sequence F
tgggcagtggatacaccattatg

SEQ ID NO: 15 - DNA, 26 bp, synthetic construct
  source 1..26: mol_type = other DNA; note = Reverse primer sequence R
ggaggaaatagcagagggaagagagc

SEQ ID NO: 16 - RNA, 22 nt, synthetic construct
  source 1..22: mol_type = other RNA; note = Sequence of miR-30d
tgtaaacatccccgactggaag

SEQ ID NO: 17 - DNA, 22 bp, synthetic construct
  source 1..22: mol_type = other DNA; note = miR-30d sequece with the uracil replaced with thymine
tgtaaacatccccgactggaag

SEQ ID NO: 18 - DNA, 30 bp, synthetic construct
  source 1..30: mol_type = other DNA; note = Sequence of scramble
ggcgggttaatcattaactacaaggaaccc

SEQ ID NO: 19 - DNA, 23 bp, synthetic construct
  source 1..23: mol_type = other DNA; note = Fragment of EGFP gene
atggtgagcaagggcgaggagct

SEQ ID NO: 20 - DNA, 21 bp, synthetic construct
  source 1..21: mol_type = other DNA; note = Fragment of EGFP gene
ggcatggacgagctgtacaag